(19) United States
(12) Patent Application Publication
(10) Pub. No.: US 2005/0177247 A1
(43) Pub. Date: Aug. 11, 2005

(54) Orthopaedic scaffolds for tissue engineering

(76) Inventors: Leigh Trevor Canham, Worcestershire (GB); Jeffery Lee Coffer, Forth Worth, TX (US); Priyabrats Mukherjee, Fort Worth, TX (US)

Correspondence Address: NIXON & VANDERHYE, PC, 901 NORTH GLEBE ROAD, 11TH FLOOR, ARLINGTON, VA, 22203, US

(21) Appl. No.: 10/516,340

(22) PCT Filed: May 29, 2003
(86) PCT No.: PCT/GB03/02364

(30) Foreign Application Priority Data
May 31, 2002 (GB) 02126670

(51) Int. Cl. A61F 2/28

(52) U.S. Cl. 623/23.51; 264/45.1; 435/396

(57) ABSTRACT

A process for preparing an orthopaedic scaffold, or other solid body, said process comprising forming shaped blocks of a bioactive material comprising silicon, treating one or more selected surfaces of said blocks such that they will adhere to a similarly treated surface of a similar block, and self-assembly of a scaffold comprising two or more of said blocks under conditions in which the treated surfaces will bind together, and thereafter recovering the assembled structure. Products including orthopaedic scaffolds obtained using this process are also provided.

The present invention relates to processes for making self-assembly orthopaedic scaffolds for tissue engineering, and to the orthopaedic scaffolds obtained thereby.

### BACKGROUND OF THE INVENTION

Tissue engineering (TE) embodies a major new trend in medicine that is helping the body to heal itself. Engineering new bone is expected to be an important TE area over the next decade since bone & cartilage are simpler cellular systems and the body already has an in-built regeneration system (“remodelling”) for bone.

The need for bone replacement can arise from trauma, infection, cancer or musculoskeletal disease. Every year, surgeons in the USA alone perform over 450,000 bone grafts. Both natural and synthetic materials are used in a variety of approaches.

A bone autograft is a portion of bone taken from another area of the skeletal system of the patient. Autografting is considered the gold standard in efficacy for procedures that require supplemental bone, but autograft harvest carries risks and considerable patient discomfort. Recovery time is slow and often exceeds 6 months.

Alternatives are bone allografts, involving a human donor source other than the recipient patient. An allogenic bone graft, commonly derived from human cadavers, is cleaned,sterilised, and stored in a bone bank prior to use. However the sterilization process may be compromise the strength of the bone, and there is a perceived risk of transmitting infectious disease. It is also known to have limited osteoconductive and osteoinductive capabilities, the importance of which is discussed more fully below.

A bone xenograft, in which processed bone from animals is transplanted to humans offers higher productivity but is perceived to be riskier than allografting in terms of disease transmission.

A range of bone graft materials have been in clinical use for some time and others are under development. Approved natural products include demineralised human bone matrix, bovine collagen mineral composites and processed coralline hydroxyapatite. Synthetic products which are approved include calcium sulphate scaffolds, bioactive glass scaffolds and calcium phosphate scaffolds. These materials are required to have a number of particular physical and biological properties.

Orthopaedic scaffolds are used as both temporary or permanent conduits for bone. They can both encourage and direct growth across a fracture site, or regrowth of damaged or infected bone. Whilst the composition of cortical and cancellous bone is very similar, their microstructure differs considerably. Compact or cortical bone contains neurovascular “Haversian” canals of about 50-100 micron width, which are held together by a hard tissue “stroma” or “interstitium”. The structure of spongy, cancellous bone differs from cortical bone in being more open-spaced and trabecular.

Any material used in an orthopaedic scaffold is required to have a porosity which closely reflects that of the bone it is intended to replace. For example, a biomimetic scaffold for cancellous bone would have a thin interstitium lattice interconnected by pores of 500-600 micron width. It is the interstitium which does not have blood within, that can be substituted by a biodegradable composite material.

In addition, in order for an implant to be used as a replacement for bone it must be capable of at least allowing osteointegration and osteoconduction. Osteointegration refers to the direct chemical bonding of a biomaterial to the surface of bone without a thick intervening layer of fibrous tissue.

An osteoconductive biomaterial passively allows living bone to grow and remodel over its surface. Normal osteoblast behaviour is thus maintained which includes mineralisation, collagen production and-protein synthesis.

Two desired further properties for an OTE scaffold material are that it is osteoinductive or osteogenic, and degradable at a rate that matches that of new bone in-growth.

An osteoinductive biomaterial actively encourages bone growth, by for example, recruiting and promoting the differentiation of mesenchymal stem cells into osteoblasts. An osteoinductive implant will often induce bone to grow in areas where it would not normally grow i.e. “ectopic” bone formation. This induction process is normally biochemical, but it could be mechanical or physical in nature. Finally, an osteogenic biomaterial is one that contains cells that can form bone or can differentiate into osteoblasts.

Typical requirements on biodegradation rates are that the scaffold maintains its structural integrity for 4-10 weeks for cartilage repair and 3-8 weeks for bone repair

The mechanical requirements of the material are highly dependant on the type of tissue being replaced. Cortical bone has a Youngs Modulus of 15-30 GPa, cancellous (spongy, trabecular) bone has a Youngs Modulus of 0.01-2GPa and cartilage has a Youngs Modulus of less than 0.001 GPa and the material used in any particular case should reflect this as far as possible.

Many approaches to fabricating porous scaffolds have been developed for biodegradable polymer systems, these include solvent casting and particulate leaching, melt moulding, fibre bonding, gas foaming or membrane lamination.

Different approaches are known for the more thermally stable ceramic systems such as hydrothermal conversion and burn-out of dispersed polymer phase.

Many of the existing porous biodegradable polymeric systems have been found to have limitations for use as orthopaedic scaffolds for cell ingrowth. For instance, it is often possible only to obtain a poor match of mechanical properties to the tissue being replaced. There is difficulty in achieving uniform porosity over large distances within the polymeric system, and although matrices can be osteoconductive, they may not have any osteoinductive ability.

Porous ceramic systems also suffer from poor control over pore size distribution, and may also have poor moldability compared to polymers.

To address some of these deficiencies, more complex scaffolds are under development, such as polymer/ceramic composites, seed polymer scaffolds with mesenchymal stem cells and biomaterial/tissue hybrid structures.

WO 98/44964 discloses biocompatible compositions comprising porous biodegradable polymer having bioactive material such as silicon compounds (silica-gel or bioactive glass) for the replacement of bone grafts.

WO 01/95952 A1 describes the use of bioactive and biodegradable silicon in orthopaedic scaffolds. In particular, silicon is shaped to the desired shape and then porosified electrochemically, to form bioactive material. A significant limitation of nanostructuring silicon via electrochemistry is the inability to anodise across the depths needed for large implants. In another embodiment, porous silicon powder is mixed with powder of a biodegradable polymer (polycaprolactone), which is melted together to form a bioactive composite for orthopaedic use. There is however no disclosure as to how large channels for bone in-growth could be realized in such composites.

The applicants have found that orthopaedic scaffolding can advantageously be prepared from materials of this type using a particular self assembly method.

### SUMMARY OF THE INVENTION

According to the present invention there is provided a method of preparing an orthopaedic scaffold, said method comprising forming shaped blocks of a bioactive material comprising silicon, treating one or more selected surfaces of said blocks such that they will adhere to a similarly treated surface of a similar block, and self-assembly of a scaffold comprising two or more of said blocks under conditions in which the treated surfaces will bind together.

As used herein, the term “blocks” refer to polygon shaped, three-dimensional structures. They may have a variety of shapes to suit the desired construction, including flat-sided polygons or spheroidal shapes with one or more planar regions. Typically they will be square, hexagonal or octagonal in cross section. Suitably, they are hollow or have a central hole. They will generally be relatively small in size, for example from 1-8 mm and preferably from 1.5-5 mm across. In particular, they will comprise cubes which are, for example 3 mm×3 mm×3 mm, or cuboids of similar dimensions in cross section but with a reduced depth for example of from 0.8 to 0.9 mm, hexagons which for example, range from 1.9 to 3.9 mm across, which a depth of 0.8 to 0.84 mm

Suitably the blocks will be at least partially porous, and preferably with a porosity in the range of from 10 to 90%, and preferably in the range of from 30 to 80%, most preferably from 35%-58%. Porosity values of from 30 to 80% can be produced for example, by introduction of 2 mm channels in 1, 2 or 3 dimensions into the block. Higher porosity values may be possible by including soluble salts into the materials used to prepare the blocks (for example a mixture of bioactive silicon powder and polymer described hereinafter), and the subsequent removal of the salt by incubation in aqueous media. This will allow it to be used in the context of the various types of bone structures described above.

Using the method of the invention, it is possible to obtain the larger scaffolds needed for most bone grafts with the desired nanostructure throughout. Furthermore, the scaffolds will have highly ordered structures. For bone grafts this translates into excellent control of macroporosity and macropore architecture

Suitably, the bioactive material used comprises bulk crystalline silicon, porous silicon, amorphous silicon or polycrystalline silicon, as well as composites of bioactive silicon and other materials, as described in WO 01/95952. In particular however, the bioactive material used in the method of the invention comprises a composite of bioactive silicon and a biocompatible polymer.

Silicon is suitably present in the composite in the form of polycrystalline or porous particles, which are fused to polymer carrier material. These are suitably formed by pre-forming the desired bioactive silicon particles, mixing these with the polymer carrier material, also in powder or granular form, and heating the resultant mixture so as to fuse the mixture. Suitably the polymer is a low melting polymer, for example with a melting point of less than 150° C. and preferably less than 100° C. so that the melting process can be carried out without losing the nanostructure of the silicon particles.

Particular examples of suitable polymers include polycaprolactone (PCL), poly(3-hydroxybutyrate (PHB), poly(lactic acid) (PLA), polyglycolic acid (PGA), polyanhydrides, polyorthoesters, polyiminocarbonates, polyphosphazenes and polyamino acids. Preferably the polymer used in the composite is PCL with a molecular weight in the range of from about 2 kD up to 15 kD product.

Silicon used in the method of the invention may be bioactive silicon, resorbable silicon or biocompatible silicon. As used herein, the term “bioactive” refers to components that bind to tissue. Resorbable silicon is defined as being silicon which dissolves over a period of time when immersed in simulated body fluid solution. “Biocompatible” refers to materials which are acceptable for at least some biological applications, and in particular may be compatible with tissue. It will be appreciated that ‘silicon’ as used herein refers to materials comprising elemental silicon, including for example semi-conducting forms of silicon.

These properties depend upon the physical form of the silicon, in particular whether it is porous, polycrystalline, amorphous or bulk crystalline and are described in more detail in WO 97/06101.

Depending upon the particular use and mode of action of the desired orthopaedic scaffold, inclusion of porous and/or polycrystalline silicon may be preferred because these nanostructured forms have been found to promote calcification and hence bone bonding. The semiconductor properties of the porous and/or polycrystalline silicon opens the way for electrical control of the treatment, repair or replacement process. Furthermore porous silicon and particularly mesoporous silicon having a pore diameter in the range of from 20 to 500 Å, and polycrystalline silicon of nanometer size grains has been found to be resorbable. Corrosion of silicon during the resorption process produces silicic acid, which is known to stimulate bone growth.

Silicon having these properties may be obtained, for example by electrolysis of silicon wafers, as described for example in WO 97/06101, as silicon nanocrystals from pyrolysis reactions, from silicon nanowires and/or as microcrystalline silicon.

The mass ratio of silicon:organic polymer in the composite is suitably in the range of from 1:99 to 99:1 and preferably from 1:20 to 1:4 w/w.

Nanostructured silicon/polymer composites are particularly preferred for use in the method of the invention since they provide good moldability combined with bioactivity. In addition, they have tunable mechanical properties for a fixed chemistry which is helpful for the regulatory process. The porosity of the blocks may be readily “tailored” to the desired porosity through physical deformation. It will in any event, be largely dependent upon the amount of composite placed in a given mold during structure fabrication, and may if desired or necessary be modified following production for example by a wet chemical etching process, or a salt incorporation followed by selective leaching.

Treatment of the selected surfaces may be carried out in various ways, provided it leads to the “activation” of the surface to binding. In particular, it produces reactive groups on the surface, which are able to react, for example with coupling agents, to form covalent bonds, which hold the blocks firmly together. Examples of such reactive groups include silanol groups (SiOH).

Treatments may be effected chemically, for example using the techniques described in WO 00/26019 or WO 00/66190. However, it is difficult to limit chemical derivatization to particular surface areas, and therefore a preferred method comprises activating the surface by exposing the surface to an activating radiation or plasma. In particular, the applicants have found that a brief exposure, for example of from 15 seconds to 1 hour or more preferably from 1-10 minutes, of the selected surfaces to oxygen-rich plasma will increase the density of silanol (Si—OH) moieties on the surface as well as etching away some of the surface polymer (where present), and so further expose the crystalline Si domains.

Alternatively, a surface of a silicon/polymer composite block may be activated for binding by selectively enriching the amount of silicon exposed at that surface of the block. This may conveniently be achieved by applying powdered silicon to the surface at a temperature sufficient to cause the polymer component to soften and adhere to the silicon.

By ‘self-assembly’ is meant binding together of individual elements by simple mixing to form a desired architecture. Thus two or more blocks can form an organized structure wherein the organization within the structure is determined, under the appropriate assembly conditions, solely by the choice of which surface(s) of the constituent blocks are treated to activate them to binding. In this way, the intricate molding processes are avoided.

Suitable coupling reagents will depend upon the form of the activation of the surface.

When using oxygen plasma as outlined above, suitable coupling agents include alkoxysilane reagents such as tetraethoxysilane (TEOS), tetramethoxysilane (TMOS), aminopropyltriethoxysilane (APTES) or mercaptopropyltrimethoxysilane (MPTS). The coupling reagent is suitably dissolved in a solvent such as water, at concentrations of from 0.0015 to 0.0132 molar. The higher the concentration of coupling agent, the greater the degree of coupling which will occur, and thus, this will affect the dimensions of the final structure which may be achieved. Pre-treated blocks are then mixed in the solution of the coupling reagent with stirring, until the desired structure has been formed. Suitably, the reaction duration and coupling reagent concentration is set so that the structure will be obtained within a period of from 5 to 30 minutes.

When the surface has been activated by selective enrichment of the amount of silicon present, a suitable method for coupling involves promoting association of activated surfaces through capillary forces and chemical cross-linking of the associated surfaces. Typically, a polysaccharide such as starch may be used to form the cross-links. Suitably, the enriched sites are coated with aqueous starch solution and the coated blocks are agitated in the presence of a mixture comprising perfluorodecalin (PFD) and hexane. The liquid may then be removed and the assembled product dried.

The selection of the surfaces which are treated depends upon the construction being produced. In order to produce essentially “one dimensional” shapes, the upper and/or lower surface of the blocks is treated. This means that when they combine together, they pile up in an essentially columnar arrangement.

For the creation of essentially two dimensional structures, side edges of the blocks are suitably treated. In this way, the blocks will pack together alongside one another. For truly three dimensional structures, at least some of each of the side and/or upper and lower surfaces will be pre-treated before the mixing process begins.

The present applicants have found that the scaffold assembly is reversible and can be disassembled. The ability of the scaffold to disassemble over a suitable period of time and at a rate which matches the rate of formation on new bone growth can be advantageous in bone grafts, for example, as discussed above.

Furthermore, by making use of these disassembling properties it is possible to obtain delayed or sustained release of a desired substance, such as a pharmaceutically active substance, by trapping molecules of the substance within the scaffold of the invention such that they can be release as the scaffold disassembles. Where reversibility of the scaffold assembly is desired, it is preferable that the scaffold is prepared using polysaccharide cross-linking of silicon-enriched blocks. If desired, once the scaffold has been prepared as described above, other surface modification reactions may be carried out to alter the biological activity or specificity. For example, APTES may be coupled to the surface, together with other small peptides, which alter vascular growth endothelial factor (VGEF) activity or other cellular recognition/adhesion in vivo.

The stability of the assembled structure may also be improved by application of heat.

The invention further comprises an orthopaedic scaffold, obtainable by a process as described above.

Thus the invention further provides an orthopaedic scaffold comprising a plurality of blocks of a bioactive material comprising silicon, adhered together. In particular the bioactive material comprises a composite of silicon and a biocompatible polymer as described above. Suitably, also, the blocks are adhered together by means of covalent bonds.

Orthopaedic scaffolds in accordance with the invention may have a variety of applications. For example, they may be used in the treatment of hip fracture, arthrosis of the hip and knee, vertebral fracture, spinal fusion, long bone fracture, soft tissue repair and osteoporosis.

The process of the invention may have wider applications, for example in the preparation of other bodies comprising silicon, and in particular medical devices or implants which are required to be bioactive. Furthermore, the formation of covalent chemical bonds between elements of a “self-assembled” polymer body has not previously been carried out. Earlier self-assembly strategies of micro/millimeter scale polymer objects have employed non-biocompatible or non-bioactive polymers (such as Poly DiMethylSiloxane (PDMS)) whose condensed long range order is made manifest by physical capillary forces. Using the method of the invention, it is possible to produce covalent chemical bonds, and particularly strong covalent interfacial bonds between blocks. This strategy may find application in the production of solid bodies for a variety of non-medical purposes as well as those listed above.

Thus in a further aspect, the invention provides a process for preparing solid object, said process comprising forming shaped blocks of a material comprising silicon, treating one or more selected surfaces of said blocks such that they will adhere to a similarly treated surface of a similar block, and combining two or more of said blocks together under conditions in which the treated surfaces will bind together, and thereafter recovering the assembled structure.

Suitably in this process, covalent chemical bonds are formed between the surfaces to bind the blocks together. Preferred options for carrying out are similar to those described above.

Still further, the invention provides a process for preparing a solid object, said process comprising forming shaped blocks of a material, treating one or more selected surfaces of said blocks such that they will adhere to a similarly treated surface of a similar block, and combining two or more of said blocks together under conditions in which the treated surfaces will form covalent chemical bonds therebetween, and thereafter recovering the assembled structure.

Again, preferred means of carrying out this process will be analogous to those described above.

### DESCRIPTION OF THE FIGURES

FIG. 1 shows typical monomer blocks of a polycaprolactone/silicon composite, which are either hexagonal (a) and of 3 mm diameter, or cuboid with a 4 mm edge length.

FIG. 2 shows one dimensional assemblies formed from the hexagonal blocks of FIG. 1, wherein (a) comprises a tetramer of hexagons, and (b) comprises a pentamer of hexagons.

FIG. 3 shows two dimensional networks comprising (a) a trimer of hollow hexagonal blocks, (b) a close packed array of solid hexagonal blocks and (c) a tile of 8 cubes.

FIG. 4 shows a three dimensional scaffold, comprising an octamer of cubes.

FIG. 5 shows an SEM image obtained along the interior of a channel in a mesoporous silicon/PCL composite cube which has been exposed to a solution of simulated body fluid (SBF).

FIG. 6 shows an assembly formed by polysaccharide coupling of silicon/PCL composite cubes in which all of the faces have been enriched with silicon. The corresponding unmodified cubes do not self-assemble under the same conditions.

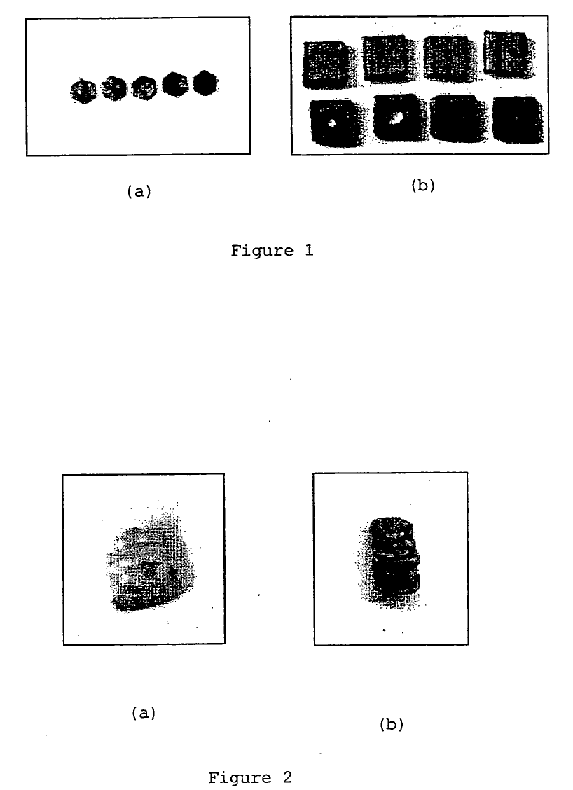

### DESCRIPTION OF THE INVENTION

### EXAMPLE 1

Step 1

Synthesis of Individual Structures:

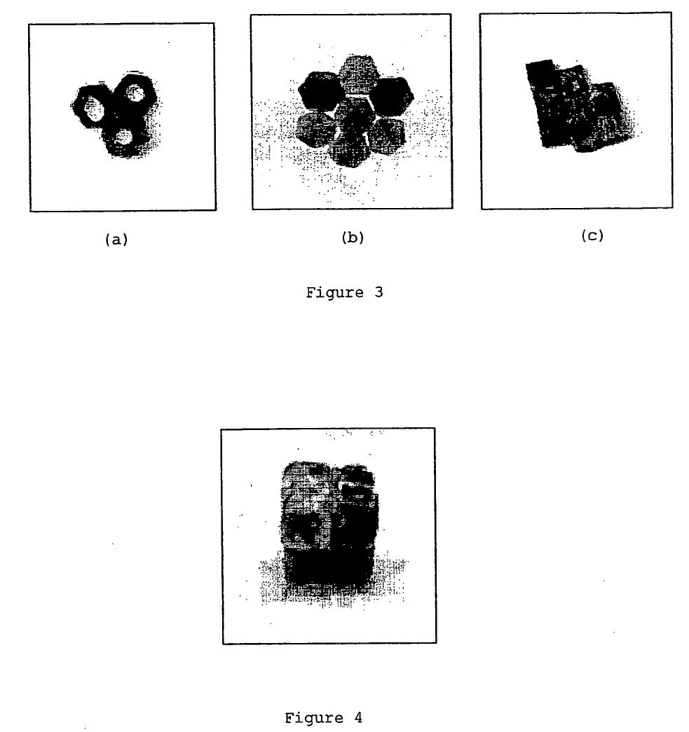

The individual composite building blocks (in the form of cubes or hexagons) were prepared by initially grinding polycaprolactone (PCL) with the porous powdered silicon material, obtained as described in WO01/95952, in various ratios by mass. The ratios prepared were as follows:

| | Mass of PCL | Mass of porous |
|---|---|---|
| Product | Powder | silicon powder |
| 1-D pentamer (FIG. 2b) | 0.3077 g | 0.0596 g |
| 2-D trimer (FIG. 3a) | 0.4181 g | 0.0827 g |
| 2-D hexamer (FIG. 3b) | 0.1652 g | 0.0338 g |
| 2-D octamer (FIG. 3c) | 0.6614 g | 0.1335 g |
| 3-D octamer (FIG. 4) | 0.6403 g | 0.1315 g |

These composite powders were then poured into pre-formed PDMS molds with the desired 2-D shape (hexagonal or square). The molds were heated in an oven at 110° C. for −1 hr, and then cooled to room temperature. The solid composite blocks obtained could then be cut to the desired thickness between 0.8 mm to 4 mm.

Step 2

Preparation of Organized Assemblies:

The 2-D octamer illustrated in FIG. 3c was prepared as follows. Predetermined surfaces of the blocks obtained in Step 1 were exposed to a brief (8 minutes long) oxygen-rich plasma in order to etch away some of the surface PCL, expose the crystalline Si domains, and increase the density of silanol (Si—OH) moieties on the surface. Eight blocks were added to a 0.0063 molar aqueous solution of MPTS together with 2.8 ml of ethanol at room temperature, and stirred for 30 minutes until the desired structure was achieved.

Other assemblies were prepared in an analogous manner. Examples of 1D, 2D and 3D assemblies prepared in this way are shown in FIGS. 2-4.

### EXAMPLE 2

Selective Enrichment of Selected Sites

Silicon powder material was spread on a rectangular glass slide. The glass slide was then placed over a hot plate and the temperature of the hot plate was adjusted to 200° C. Selected sites of composite building blocks (in the form of cubes or hexagons) prepared as described above were touched carefully with the hot silicon powder. The portion of the PCL polymer in contact with the hot silicon softened, leading to incorporation of the silicon material at those selected sites.

### EXAMPLE 3

Calcification of BioSilicon Embedded in a Hollow PCL Cube

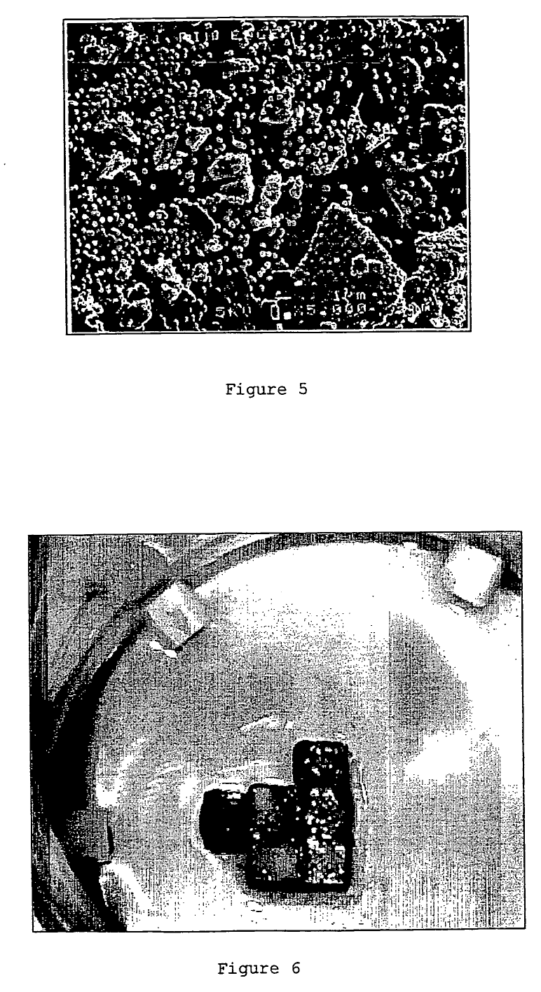

A composite structure composed of 11.4% mesoporous Si (w/w) was prepared by a method analogous to Example 1 and exposed to a solution of SBF at 37° C. for 14 days. Scanning electron microscopy was then used to examine the interior of a one dimensional channel in the structure. The image (FIG. 5) clearly showed numerous calcified deposits, the composition of which was confirmed in the corresponding energy dispersive x-ray spectrum. This result is in stark contrast to a control sample composed solely of PCL, where an absence of calcified deposits was evident on the surface of the material.

### EXAMPLE 4

Polysaccharide Coupling of Composite Blocks

After selective face (or edge) enrichment with silicon powder as described in Example 2 above, the silicon-enriched sites were coated with an aqueous solution of starch (2%) prior to the assembly process according to the following general procedure (described here for a 2-dimensional assembly process):

Three opposite (1,3) face-modified cubes were placed in a 50 ml beaker (diameter 4.0 mm) containing 15.0 ml PFD and 10.0 ml n-hexane, rotating in an orbital shaker at a speed of 200 rpm. To obtain linear chains of longer chain length, a larger vessel (800 ml beaker) containing 50 ml PFD and 50 ml n-hexane rotating in the orbital shaker with a speed of 90.0 rpm was employed. Once the assembly process was over, the liquid was removed and the assembled product was dried overnight in air at room temperature.

FIG. 6 shows the results of an experiment to compare the effect of silicon enrichment on the coupling of composite silicon/PCL blocks in the presence of starch as cross-linking agent. Six cubes (all faces silicon enriched, seen in dark in the figure) were coated with starch according to the method above and were found to assemble together to form a scaffold. By contrast, unmodified cubes (which did not have surfaces which had selectively been enriched with silicon, seen as the light cubes in the figure) did not self-assemble under the same conditions.

### EXAMPLE 5

Substance Release from a Starch-Linked PCL/Silicon Composite Structure

The ability of a PCL/silicon composite to release a substance upon cleavage of the starch-linked silicon interface was assessed by monitoring the appearance of a sensitive chromophore (Tris (2,2-bipyridyl)ruthenium(II) Chloride) in aqueous solution.

Two cubes (each with a spherical cavity at one face; mass 0.0492 g) were embedded with the Ru complex (˜0.4 mg) and silicon crystals were then embedded at the periphery of the mouth of each cavity (0.4 mg). Dilute starch solution was added to each silicon-rich surface and the structure was assembled. The assembled structure was dried for 1 h in air and then dropped into a water/PFD mixture (12 ml PFD and 10.0 ml water) in a 50 ml beaker with a shaking rate of 216 rpm. The release kinetics were monitored up to 22 h.

The dimer was found to break up completely by 2.5 h, indicating that the cross-linking is reversible.

### EXAMPLE 6

Biological Testing

Scaffolds obtained using the method of the invention may be tested to determine their precise properties. In particular, the calcification activity, the silicon dissolution kinetics and the phase behavior at the polymer/Si interface (blending or separation—direct visualization of morphology) as well as the mechanical strength can be tested using conventional methods.

By varying the process parameters, such as the nature of the bioactive material and particularly the composite material, the size and shape of the blocks, the concentration of the coupling reagent and the length of time the blocks are immersed in it, a wide variety of orthopaedic scaffolds suitable for different purposes may be obtained.

1. A process for preparing an orthopaedic scaffold, said process comprising forming shaped blocks of a bioactive material comprising silicon, treating one or more selected surfaces of said blocks such that they will adhere to a similarly treated surface of a similar block, self-assembly of a scaffold comprising two or more of said blocks under conditions in which the treated surfaces will bind together, and thereafter recovering the assembled structure.

2. A process according to claim 1 wherein the said blocks are square or hexagonal in cross section.

3. A process according to claim 1 or claim 2 wherein the blocks will be at least partially porous.

4. A process according to claim 1 wherein the bioactive material comprises bulk crystalline silicon, amorphous silicon, porous silicon, polycrystalline silicon, or a composite of bioactive silicon and another material.

5. A process according to claim 4 wherein the bioactive material is a composite of bioactive silicon and a biocompatible polymer.

6. A process according to claim 5 wherein the composite is obtained by mixing bioactive silicon particles with a polymer in powder or granular form, and heating the resultant mixture so as to fuse it.

7. A process according to claim 6 wherein the mixture is heated in a mold to form a block of a desired shape.

8. A process according to claim 6 wherein the polymer has a melting point of less than 150° C.

9. A process according to claim 5 wherein the biocompatible polymer is polycaprolactone.

10. A process according to claim 5 wherein the mass ratio of silicon: organic polymer in the composite is from 1:99 to 99:1.

11. A process according to claim 10 wherein the mass ratio of silicon: organic polymer is in the range of from 1:20 to 1:4 w/w.

12. A process according to claim 1 wherein the surfaces bind together by forming covalent chemical bonds therebetween.

13. A process according to claim 1 wherein the said one or more surfaces of the blocks are treated so as to increase the density of silanol groups (SiOH) thereon.

14. A process according to claim 13 wherein the said one or more surfaces are exposed to an oxygen-rich plasma, and thereafter mixed with similarly treated blocks in the presence of a coupling agent.

15. A process according to claim 14 wherein the coupling agent is an alkoxysilane.

16. A process according to claim 15 wherein the alkoxysilane is in aqueous solution.

17. A process according to claim 4 wherein the said one of more surfaces of the blocks are treated so as to enrich the amount of silicon exposed thereon and thereafter mixed with similarly treated blocks in the presence of a coupling agent.

18. A process according to claim 17 wherein the coupling agent is a polysaccharide.

19. A process according to claim 18 wherein the coupling agent is a starch.

20. A process according to claim 1 wherein the surface of the assembled structure is treated to alter its biological activity.

21. A process according to claim 1 wherein the assembled structure is heated to raise its mechanical strength.

22. An orthopaedic scaffold comprising a plurality of blocks of a bioactive material comprising silicon, adhered together.

23. An orthopaedic scaffold according to claim 22 wherein the bioactive material comprises a composite of silicon and a biocompatible polymer.

24. An orthopaedic scaffold according to claim 22 wherein the blocks are adhered together by means of covalent bonds.

25. A process for preparing solid object, said process comprising forming shaped blocks of a material comprising silicon, treating one or more selected surfaces of said blocks such that they will adhere to a similarly treated surface of a similar block, and self-assembly of a structure comprising two or more of said blocks under conditions in which the treated surfaces will bind together, and thereafter recovering the assembled structure.

26. A process according to claim 23, wherein covalent chemical bonds are formed between the surfaces to bind the blocks together.

27. A process for preparing solid object, said process comprising forming shaped blocks of a material, treating one or more selected surfaces of said blocks such that they will adhere to a similarly treated surface of a similar block, and self-assembly of a structure comprising two or more of said blocks under conditions in which the treated surfaces will form covalent chemical bonds therebetween, and thereafter recovering the assembled structure.

28. A process for preparing an orthopaedic scaffold substantially as hereinbefore described with reference to the Examples.
